(19) United States
(12) Patent Application Publication
(10) Pub. No.: US 2018/0221282 A1
(43) Pub. Date: Aug. 9, 2018

(54) METHODS FOR MAKING PHARMACEUTICAL SOLID DOSAGE FORMS OF SPRAY-DRIED DISPERSIONS

(71) Applicants: Yung-Chi LEE, Kenilworth, NJ (US); David HARRIS, Kenilworth, NJ (US); Justin David MOSER, West Point, PA (US); Michael MCNEVIN, West Point, PA (US); Amber BROADBENT, Bend, OR (US); Robert HALL, Bend, OR (US); Ari ERICSON, Bend, OR (US); Merck Sharp & Dohme Corp., Rahway, NJ (US)

(72) Inventors: Yung-Chi Lee, New Providence, NJ (US); David Harris, New Providence, NJ (US); Justin David Moser, Collegeville, PA (US); Michael McNevin, Doylestown, PA (US); Amber Broadbent, Bend, OR (US); Robert Hall, Bend, OR (US); Ari Ericson, Fontana, WI (US)

(73) Assignee: Merck Sharp & Dohme Corp., Rahway, NJ (US)

(21) Appl. No.: 15/749,934

(22) PCT Filed: Jul. 29, 2016
(86) PCT No.: PCT/US16/44604

§ 371 Date: Feb. 2, 2018

Related U.S. Application Data
(60) Provisional application No. 62/200,707, filed on Aug. 4, 2015.

(51) Int. Cl. A61K 9/16; A61K 9/10; A61K 31/5365; A61K 9/20

(52) U.S. Cl. CPC A61K 9/1694; A61K 9/10; A61K 31/5365; A61K 9/2009; A61K 9/1611; A61K 9/2095; A61K 9/2054; A61K 9/1652

(57) ABSTRACT

A process for drying a spray dried dispersion, comprising: a) providing a spray-dried dispersion comprising particles wherein the particles comprise an active agent and a polymer and optionally one or more surfactants, the dispersion having an average particle diameter of less than about 100 m; b) blending an amount of silicon dioxide with the dispersion to form a dispersion-silicon dioxide blend, wherein the amount of silicon dioxide relative to the amount of dispersion is between about 0.5 and 2.0% by weight; c) drying the blend with a secondary dryer.

### BACKGROUND

The process of spray drying fluids tends to generate powder with 1) residual solvent higher than International Conference on Harmonization of Technical Requirements for Registration of Pharmaceuticals for Human Use (ICH) limitations during the primary spraying step, and 2) a low bulk powder density which causes posor flowability. A secondary drying step is used to remove the residual solvent below the ICH limitation before the spray-dried powder can be further processed into a finished drug product such as a capsule and tablet. To effectively remove the residual solvent with minimum open powder handling, reducing powder exposure to the worker during operation, a tumbling heated vessel, such as an Ekato dryer, under vacuum, is commonly applied used for a secondary drying step. However, due to the poor flowability of the spray dried powder, resulting yield of processed powder can be significantly reduced if the tumbling heated vessel is used in the secondary drying step.

### SUMMARY OF THE INVENTION

The invention is a process for drying a spray dried dispersion, comprising:

a) providing a spray-dried dispersion comprising particles wherein the particles comprise an active agent and a polymer and optionally one or more surfactants, the dispersion having an average particle diameter of less than about 100 μm;

b) blending an amount of silicon dioxide with the dispersion to form a dispersion-silicon dioxide blend, wherein the amount of silicon dioxide relative to the amount of dispersion is between about 0.5 and 2.0% by weight;

c) drying the blend with a secondary dryer.

In one embodiment, the active agent is dimethyl ((25,2′S)-((25,2′S)-((6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-1,2-diyl))dicarbamate or dimethyl N,N′-([(6S)-6-phenylindolo[1,2-c][1,3 ]benzoxazine-3,10-diyl]bis {1H-imidazole-5,2-diyl-(2S)-pyrrolidine-2,1-diyl [(2S)-3-methyl-l-oxobutane-1,2-diyl]})dicarbamate.

In one embodiment, a second amount of silicon dioxide is added to the dispersion- silicon dioxide blend during step c), wherein the second amount of silicon dioxide relative to dispersion is between about 0.5 and 1.5% by weight.

In another embodiment the secondary dryer is an agitation dryer or vertical dryer.

In another embodiment the vertical dryer is a vacuum contact dryer.

The invention is also a process for forming a pharmaceutical tablet, which comprises:

a) providing a spray-dried dispersion comprising particles wherein the particles comprise an active agent and a polymer and optionally one or more surfactants, the dispersion having an average particle diameter of less than about 100 μm;

b) blending an amount of silicon dioxide with the dispersion to form a dispersion-silicon dioxide blend, wherein the amount of silicon dioxide relative to the amount of dispersion is between about 0.5 and 2.0% by weight;

c) drying the dispersion-silicon dioxide blend with a secondary dryer;

d) adding pharmaceutically acceptable excipients to the dispersion- silicon dioxide blend; and

e) forming a tablet,

wherein the active agent is dimethyl ((2S,2′S)-((2S,2′S)-((6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-1,2-diyl))dicarbamate or dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate.

In one embodiment, the active agent in step a) is a first active agent, and a second active agent is added to the blend in step d).

In one embodiment, the active agent in step a) is a first active agent, a second active agent is added to the blend in step d), and a third active agent is added to the blend in step d).

In one embodiment, following step d), the process additionally includes granulating together the added pharmaceutically acceptable excipients and dispersion-silicon dioxide blend.

### DETAILED DESCRPITION OF THE INVENTION

As used herein, the terms “active agent” and “active ingredient” mean a drug, medicament, pharmaceutical, therapeutic agent, nutraceutical, nutrient, or other compound. The active agent may be a “small molecule,” generally having a molecular weight of 2000 Daltons or less. The active agent may also be a “biological active.” Biological actives include proteins, antibodies, antibody fragments, peptides, oligonucleotides, vaccines, and various derivatives of such materials. In one embodiment, the active agent is a small molecule. In another embodiment, the active agent is a biological active. In still another embodiment, the active agent is a mixture of a small molecule and a biological active. In yet another embodiment, the compositions made by certain of the disclosed processes comprise two or more active agents.

As used herein, the term “excipient” means a substance that may be beneficial to include in a composition with an active agent. The term “excipient” includes inert substances as well as functional excipients that may result in beneficial properties of the composition. Exemplary excipients include but are not limited to polymers, glidants, sugars, salts, buffers, fats, fillers, disintegrating agents, binders, surfactants, high surface area substrates, flavorants, carriers, matrix materials, and so forth. Specific examples can include microcrystalline cellulose, lactose monohydrate, croscarmellose sodium, magnesium stearate, and derivatives and mixtures thereof.

As used herein, the term “glidant” means a substance that, when added to a powder, improves the flowability of the powder, such as by reducing inter-particle friction. Exemplary glidants include but are not limited to colloidal silicas, colloidal silicon dioxide, fumed silica, CAB-O-SIL® M-5P, AEROSIL®, talc, starch, and magnesium aluminum silicates.

Following spray-drying, the secondary drying process step of the invention using silicon dioxide removes residual solvents to meet regulatory limitations, such as 5000 ppm for acetone and 3000 ppm for methanol, and removes water to improve physical stability. A secondary dryer, such as an agitation dryer, e.g., Ekato dryer, under vacuum and temperature control, offers several advantages over other dryers, such as an oven tray dryer. An agitation dryer presents a closed system that is less sensitive to environment humidity conditions. Drying efficiency is higher than an oven tray dryer because the powder bed is constantly agitated under vacuum. Finished powder can be transferred under the closed system and thereby reduce powder exposure of workers in an operation area.

Spray drying

Spray drying involves transformation of a formulation from a fluid state into a dried form by spraying the formulation into a hot drying medium. The formulation can be either a solution, suspension, or a paste. The spray dried product is typically in the form of a powder consisting of single particles or agglomerates, depending upon the physical and chemical properties of the formulation and the dryer design and operation The basic technique includes the following four steps: a) atomization of the formulation solution into a spray; b) spray-gas contact; c) drying of the spray; and d) separation of the dried product from the drying gas.

The invention provides methods and formulations for providing a spray dried product that can be used to fill capsule preparations or to manufacture tablets. The pharmaceutical solution to be spray dried is preferably selected so as to provide (upon spray drying) a substantially uniform powder with a favorable moisture or solvent content and dissolution profile.

The process for spray drying a pharmaceutical formulation is preferably undertaken aseptically. Prior to the spray drying step, a solution compounding system may be used to rapidly mix the active pharmaceutical ingredient (API) (also referred to as an “active ingredient” or “active agent”) and excipients in an environmentally controlled system, including one or more polymers e.g., polyvinyl pyrrolidone (PVP), polyethyleneoxide (PEO), poly(vinyl pyrrolidone-co-vinyl acetate) such as Kollidon® VA 64, polymethacrylates, polyoxyethylene alkyl ethers, polyoxyethylene castor oils, polycaprolactam, poly lactic acid, polyglycolic acid, poly (lactic-glycolic) acid, lipids, cellulose, 5 pullulan, dextran, maltodextrin, hyaluronic acid, polysialic acid, chondroitin sulfate, heparin, fucoidan, pentosan polysulfate, spirulan, hydroxypropyl methyl cellulose (HPMC), hydroxypropyl cellulose (HPC), carboxymethyl ethylcellulose (CMEC), hydroxypropyl methylcellulose acetate succinate (HPMCAS), cellulose acetate phthalate (CAP), cellulose acetate trimellitate (CAT), ethyl cellulose, cellulose acetate, cellulose butyrate, cellulose acetate butyrate, dextran polymer, and pharmaceutically acceptable forms, derivatives, and mixtures thereof.

The system can be a conventional tank and mixer blending system and should minimize operator exposure to the API, and minimize the API's exposure to air/oxygen during the handling, mixing and holding of ingredients prior to spray drying in order to avoid premature degradation of the API if susceptible to oxidation. The compounded solution is spray dried and then collected into a container. A secondary drying process is applied to remove residual solvent to meet the ICH limitation. The dried powder is then transferred to a finished product, such as a capsule or tablet, with additional pharmaceutical operations and pharmaceutically acceptable fillers.

The process may utilize pre-cooled, nitrogen-purged solvent for injection that is added to a nitrogen purged tank through a flow meter to measure the quantity of solvent. Since the process may be weight based, the full quantity of solvent, which will be based on the weight of the API to be mixed, can be charged initially.

Once compounding activities and addition of other ingredients has been completed and dissolved, the solution is transferred to the spray dryer. The actual spray drying involves the atomization of a liquid solution (feedstock) into a spray of droplets, and contacting the droplets with hot gas in a drying chamber. The droplets can be produced by, for example, nozzle atomizers. Evaporation of moisture from the produced droplets and formation of dry particles proceed under controlled temperature and gas flow conditions. When the droplets are small enough and the chamber large enough, the droplets dry before they reach the wall of the chamber. The resulting product is collected as a free-flowing material. Powder is discharged continuously from the drying chamber. Operating conditions and spray dryer design are selected according to the drying characteristics of the product and powder specification.

Spray dryers generally include a feedstock pump, an atomizer, a gas heater, a gas disperser, a drying chamber, and systems for exhaust gas cleaning and powder recovery. An example spray drying system includes drying gas introduced into a pre-filter. In one aspect, the drying gas is nitrogen, and avoids the presence of oxygen. The drying gas then passes through a fan and a heater, which may be an electric heater. The drying gas then passes through an inlet gas temperature gauge monitors the inlet gas temperature before it is introduced into a drying chamber via a ceiling gas dispenser. Redundant filtration may be employed to ensure product quality.

The formulation, from the tank and mixer blending system, undergoes a filtration process prior to being fed into the drying chamber and atomized by an atomizer. The atomizer may be any type of known atomizer that allows for aseptic processing such as a pressure nozzle or a two-fluid nozzle (e.g., available from GEA Process Engineering Inc., Columbia, Md., formerly known as Niro, Inc.). The atomizer disperses the liquid formulation into a controlled drop size spray. In one particular embodiment, the atomizer is operated with a nozzle protection of 80 kg/hour nitrogen gas at 80° C. The spray is then heated in the drying chamber, The heated drying gas evaporates the liquid from the spray and forms dry particles.

After the solution has been atomized and heated, the dry particles exit the drying chamber and proceed into a cyclone, which separates the powder from the gas. The powder flows out of the cyclone at outlet into a powder collection vessel and the rest of the gas flows out past an outlet gas temperature gauge toward a cartridge filtration system. The cartridge filtration system removes fine particles at outlet. The remaining dried gas then flows through a second filter (e.g. a sterile filter), and in some embodiments through a third filter, and then back into the drying gas supply at. In one embodiment, a vortex eliminator may be used near the bottom of the cyclone to eliminate hot gas from passing through the outlet.

In various embodiments, the spray dryer may utilize a drying gas having an inlet temperature of about 60° C.-180° C., and preferably of about 90° C.-150° C. The drying gas may have an outlet temperature of about 20° C.-70° C., and preferably of about 30° C.-60° C. The nitrogen gas flow rate can range from 650-750 kg/hour but other flow rates can be used to accommodate the rate of the feedstock, equipment and temperature variations.

### Silicon Dioxide

Silicon dioxide, also known as silica, is most commonly found in nature as quartz, as well as in various living organisms. In many parts of the world, silica is the major constituent of sand. Silica is one of the most complex and abundant families of materials, and exists as several minerals as well as synthetically. Examples include fused quartz, crystal, fumed silica, silica gel, and aerogels. Applications range from structural materials to microelectronics to components used in the food industry. CAB-O-SIL® fumed silica colloidal silicon dioxide is a silicon dioxide product that is commercially available from Cabot Corporation (Boston, Mass.).

### Secondary Drying

In one embodiment, the secondary dryer is an agitation dryer or vertical dryer. In one embodiment, the vertical dryer is a vacuum contact dryer. Suitable vacuum contact dryers include but are not limited to EKATO vertical dryer SOLIDMIX VPT 400 and SOLIDMIX VST 2500 (EKATO Systems GmbH, Schopfheim, Germany). Vacuum contact dryers are suitable for drying free-flowing solids and bulk solids. Product moves upwards near the dryer wall and downwards at the center.

In one embodiment, the secondary dryer is a vacuum tumbling dryer (e.g., PB 250, PB 1000 (Jaygo Incorporated, Randolph, N.J.)).

In one embodiment, the secondary dryer is a fluidized bed dryer (e.g., a Vibro-Bed™ fluid bed dryer (Kason Corporation, Millburn, N.J.).

In one embodiment, secondary dryers suitable for use in the process of the present invention have process batch working capacity volumes of material of about 3L to about 1000L, as well as larger volume commercially available dryers.

Dispersion-silicon dioxide blends dried with a secondary dryer may be used with various excipients and glidants to form pharmaceutical dosage forms such as tablets and capsules for delivery of the active agent to the patient.

### Tablets, Capsules and Other Dosage Forms

Dispersion-silicon dioxide blends containing an active agent and dried with a secondary dryer according to the procedure of the invention can be used to prepare pharmaceutical dosage forms for delivery of the active agent to a patient. In addition to dispersion-silicon dioxide blends containing an active agent, additional active agents, including one or more, either also prepared according to the procedure of the invention, or prepared without the spray-drying or secondary drying step described herein, can be incorporated into the pharmaceutical dosage form, to create fixed dosage forms have multiple active ingredients.

Pharmaceutical compositions encompass both bulk composition and individual dosage units comprised of more than one (e.g., two) pharmaceutically active agents such as, for example, an active agent contained in a dispersion prepared according to the procedure of the invention, and an additional active agent, along with pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit, such as a tablet, capsule, pill or like unit. Pharmaceutical compositions prepared according to the procedure of the invention may be administered to mammals, including humans, either alone or, in combination with pharmaceutically acceptable carriers, excipients or diluents, in a pharmaceutical composition, according to standard pharmaceutical practice. The compounds can be administered orally or parenterally, including by intravenous, intramuscular, intraperitoneal, subcutaneous, rectal and topical routes of administration.

Pharmaceutical compositions containing active agents may be in a form suitable for oral use, for example, as tablets, troches, lozenges, aqueous or oily suspensions, dispersible powders or granules, emulsions, hard or soft capsules, syrups or elixirs. Compositions including active agents prepared by the process of the invention and intended for oral use may be prepared according to any method known to the art for the manufacture of pharmaceutical compositions, and such compositions may contain one or more additional agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active agent is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active agent in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active agent in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Active ingredient preparations prepared according to the invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

Active ingredient preparations prepared according to the invention can also be used to prepare topical dosage forms such as creams, ointments, jellies, solutions or suspensions.

Active ingredient preparations prepared according to the invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Active ingredient preparations prepared according to the invention may also be delivered as a suppository employing bases such as cocoa butter, glycerinated gelatin, hydrogenated vegetable oils, mixtures of polyethylene glycols of various molecular weights and fatty acid esters of polyethylene glycol.

When the dosage form prepared with an active ingredient preparation prepared according to the invention is administered to a human subject, the daily dosage will normally be determined by the prescribing physician with the dosage generally varying according to the age, weight, and response of the individual patient, as well as the severity of the patient's symptoms.

The dosage regimen utilizing the dosage forms can be selected in accordance with a variety of factors including type, species, age, weight, sex and the type of cancer being treated; the severity of the condition to be treated; the route of administration; the renal and hepatic function of the patient; and the particular active agent or salt thereof employed. An ordinarily skilled physician or veterinarian can readily determine and prescribe the effective amount of the drug required to treat, for example, to prevent, inhibit (fully or partially) or arrest the progress of the disease. Active agents can be administered in a total daily dose of 10 mg to 3000 mg. For example, active agents can be administered in a total daily dose of up to 3000 mg. Active agents can be administered once daily (QD), or divided into multiple daily doses such as twice daily (BID), and three times daily (TID). Active agents can be administered at a total daily dosage of up to 3000 mg, e.g., 200 mg, 300 mg, 400 mg, 600 mg, 800 mg, 1000 mg, 2000 mg or 3000 mg, which can be administered in one daily dose or can be divided into multiple daily doses as described above.

In addition, the administration can be continuous, i.e., every day, or intermittently. The terms “intermittent” or “intermittently” as used herein means stopping and starting at either regular or irregular intervals. For example, intermittent administration of an active agent may be administration one to six days per week or it may mean administration in cycles (e.g. daily administration for two to eight consecutive weeks, then a rest period with no administration for up to one week) or it may mean administration on alternate days. The active agent may be administered discontinuously rather than continuously during the treatment cycle. Thus, the active agent may be administered daily for one or more weeks during the cycle and discontinued for one or more weeks during the cycle, with this pattern of administration repeating during the treatment cycle (e.g., administration for a week and then discontinued for a week). This discontinuous treatment may also be based upon numbers of days rather than a full week. The number of days (or weeks) that the compounds of this invention are not dosed do not have to equal the number of days (or weeks) wherein the active agent is dosed. Usually, if a discontinuous dosing protocol is used, the number of days or weeks that the active agent is dosed is at least equal to or greater than the number of days or weeks that the active agent is not dosed.

In addition, the active agent may be administered according to any of the schedules described above, consecutively for a few weeks, followed by a rest period. For example, the active agent may be administered according to any one of the schedules described above from two to eight weeks, followed by a rest period of one week, or twice daily at a dose of 100-500 mg for three to five days a week. In another particular embodiment, the active agent may be administered three times daily for two consecutive weeks, followed by one week of rest.

Any one or more of the specific dosages and dosage schedules of the active agent may also be applicable to any one or more separately formulated additional active agents to be used in the combination treatment (hereinafter referred to as the “second active agent” or “third active agent”).

Moreover, the specific dosage and dosage schedule of the additional active agents can further vary, and the optimal dose, dosing schedule and route of administration will be determined based upon the specific additional active agents being used.

The route of administration of active ingredient preparations prepared according to the invention is independent of the route of administration of the other active agents. In an embodiment, the administration for active ingredient preparations prepared according to the invention is oral administration. In another embodiment, the administration for active ingredient preparations prepared according to the invention is intravenous administration. Thus, in accordance with these embodiments, active ingredient preparations prepared according to the invention are administered orally or intravenously, and the other active agents can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

In addition, active ingredient preparations prepared according to the invention and other active agents may be administered by the same mode of administration, i.e., both agents administered e.g., orally, intravenously, etc.. However, it is also within the scope of the present invention to administer active ingredient preparations prepared according to the invention by one mode of administration, e.g., oral, and to administer the other active agents by other modes of administration, e.g., intravenously, or any other ones of administration modes described hereinabove.

The first treatment procedure, administration of active ingredient preparations prepared according to the invention, can take place prior to the second treatment procedure, i.e., the second active agent, after the treatment with the second active agent, at the same time as the treatment with the second active agent, or a combination thereof. For example, a total treatment period can be decided for active ingredient preparations prepared according to the invention. The second active agent can be administered prior to onset of treatment with active ingredient preparations prepared according to the invention or following treatment with active ingredient preparations prepared according to the invention. In addition, treatment can be administered during the period of administration of active ingredient preparations prepared according to the invention but does not need to occur over the entire treatment period of active ingredient preparations prepared according to the invention.

### Granulation

In the pharmaceutical industry, granulation refers to the act or process in which primary powder particles are made to adhere to form larger, multiparticle entities called granules. It is the process of collecting particles together by creating bonds between them. Bonds are formed by compression or by using a binding agent. Granulation is extensively used in the manufacturing of tablets and pellets (or spheroids).

Granulation is carried out for various reasons, one of those is to prevent the segregation of the constituents of powder mix. Segregation is due to differences in the size or density of the component of the mix. Normally, the smaller and/or denser particles tend to concentrate at the base of the container with the larger and/or less dense ones on the top. An ideal granulation will contain all the constituents of the mix in the correct proportion in each granule and segregation of granules will not occur.

The granulation process combines one or more powder particles and forms a granule that will allow tableting or spheronization process to be within required limits. This way predictable and repeatable process is possible and quality tablets or pellets can be produced using tabletting or spheronization equipment. Many powders, because of their small size, irregular shape or surface characteristics, are cohesive and do not flow well. Granules produced from such a cohesive system will be larger and more isodiametric, both factors contributing to improved flow properties.

Some powders are difficult to compact even if a readily compactable adhesive is included in the mix, but granules of the same powders are often more easily compacted. This is associated with the distribution of the adhesive within the granule and is a function of the method employed to produce the granule. For example, if one were to make tablets from granulated sugar versus powdered sugar, powdered sugar would be difficult to compress into a tablet and granulated sugar would be easy to compress. Powdered sugar's small particles have poor flow and compression characteristics. These small particles would have to be compressed very slowly for a long period of time to make a worthwhile tablet. Unless the powdered sugar is granulated, it could not efficiently be made into a tablet that has good tablet characteristics such as uniform content or consistent hardness.

In wet granulation, granules are formed by the addition of a granulation liquid onto a powder bed which is under the influence of an impeller (in a High shear granulator, screws (in a twin screw granulator) or air (in a fluidized bed granulator). The agitation resulting in the system along with the wetting of the components within the formulation results in the aggregation of the primary powder particles to produce wet granules. The granulation liquid (fluid) contains a solvent which must be volatile so that it can be removed by drying, and be non-toxic. Typical liquids include water, ethanol and isopropanol either alone or in combination. The liquid solution can be either aqueous based or solvent based. Aqueous solutions have the advantage of being safer to deal with than solvents. The fluidized bed system provides a bed of solid particles with a stream of air or gas passing upward through the particles at a rate great enough to set them in motion. As the air travels through the particle bed, it imparts unique properties to the bed. For example, the bed behaves as a liquid. It is possible to propagate wave motion, which creates the potential for improved mixing. In a bubbling fluidized bed, no temperature gradient exists within the mass of the fluidized particles. This isothermal property results from the intense particle activity in the system. Thus, the fluid bed can be used to dry the wet product, agglomerate particles, improve flow properties, instantize the product, or produce coated particles for controlled release or taste masking. Modular systems designed to carry out multiple processes in which only a container change is necessary to change the type of unit operation being performed have been developed by all the manufacturers of fluid bed processors.

Water mixed into the powders can form bonds between powder particles that are strong enough to lock them together. However, once the water dries, the powders may fall apart. Therefore, water may not be strong enough to create and hold a bond. In such instances, a liquid solution that includes a binder (pharmaceutical glue) is required. Povidone, which is a polyvinyl pyrrolidone (PVP), is one of the most commonly used pharmaceutical binders. PVP is dissolved in water or solvent and added to the process. When PVP and a solvent/water are mixed with powders, PVP forms a bond with the powders during the process, and the solvent/water evaporates (dries). Once the solvent/water has been dried and the powders have formed a more densely held mass, then the granulation is milled. This process results in the formation of granules. The process can be very simple or very complex depending on the characteristics of the powders, the final objective of tablet making, and the equipment that is available. In the traditional wet granulation method the wet mass is forced through a sieve to produce wet granules which are subsequently dried.

The dry granulation process is used to form granules without using a liquid solution because the product granulated may be sensitive to moisture and heat. Forming granules without moisture requires compacting and densifying the powders. In this process the primary powder particles are aggregated under high pressure. Sweying granulator or high shear mixer-granulator can be used for the dry granulation.

Dry granulation can be conducted under two processes; either a large tablet (slug) is produced in a heavy duty tabletting press or the powder is squeezed between two counter-rotating rollers to produce a continuous sheet or ribbon of materials (roller compactor, commonly referred to as a chilsonator).

When a tablet press is used for dry granulation, the powders may not possess enough natural flow to feed the product uniformly into the die cavity, resulting in varying degrees of densification. The roller compactor (granulator-compactor) uses an auger-feed system that will consistently deliver powder uniformly between two pressure rollers. The powders are compacted into a ribbon or small pellets between these rollers and milled through a low-shear mill. When the product is compacted properly, then it can be passed through a mill and final blend before tablet compression.

### EXAMPLE 1

Spray dried dispersions of dimethyl ((2S,2′S)-((25,2′S)-((6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-1,2-diyl))dicarbamate (1-1)

Dimethyl ((25,2′S)-((25,2′S)-((6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-1,2-diyl))dicarbamate, which is shown below as Compound (1-1)

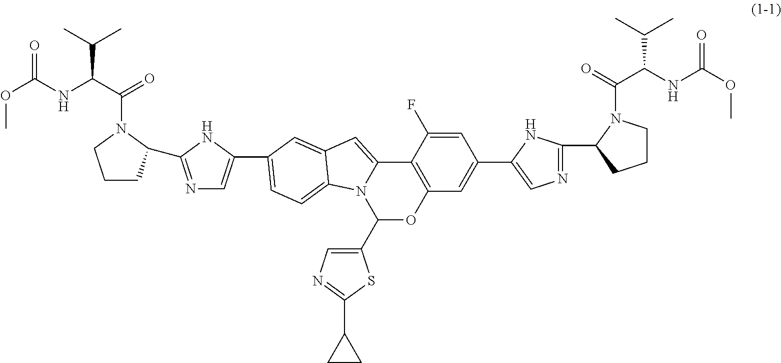 [SMILES: COC(=O)N[C@H](C(=O)N1CCC[C@H]1c1ncc(-c2cc(F)c3c(c2)OC(c2cnc(C4CC4)s2)n2c-3cc3cc(-c4cnc([C@@H]5CCCN5C(=O)[C@@H](NC(=O)OC)C(C)C)[nH]4)ccc32)[nH]1)C(C)C]

is described in WO2014/110705. Details for preparing the compound are described in Example 12 (compound 10).

Acetone, purified water, Vitamin E PGS (d-alpha tocopheryl polyethylene glycol 1000 succinate), Compound (1-1) and Hypromellose 2910 polymer were sequentially added and dissolved in a stainless steel tank equipped with agitation equipment at a temperature between 15 and 27° C. The weight proportion of the Compound (1-1)/Hypromellose 2910 polymer/Vitamin E PGS components in the blend was 20/70/10.

### Spray Drying

Using a Niro PSD-2 spray drying system with DPH Gas Disperser Type and Nozzle-Centering Device and Spraying Systems SK 76-17 (60 degree cone angle) pressure nozzle, and 12-inch outer diameter (O.D.) cyclone Product Collection, gas flow rate@ 450 kg/hr; Tᵢₙ@115° C.; Tₒᵤₜ@57° C.; Cond@−10° C.; feed pressure@485 PSI; feed rate@ 25 kg/hr, a solution feed filter of ≤250 μm, and a 75/25 (w/w) acetone/water for system warm-up and shutdown, the solvent blend described above containing Compound (1-1) was agitated for at least 30 minutes, pumped to the nozzle and spray-dried at standard operating conditions to create spray dried particles of the blend.

### Secondary Drying

A total of 352.6 grams of the mixture of spray dried particles and CAB-O-SIL® fumed silica M5P colloidal silicon dioxide was subjected to secondary drying. A one half portion of the spray dried particles were blended for one minute with 1.5 wt % CAB-O-SIL® fumed silica M5P colloidal silicon dioxide in a volume ratio of particles to CAB-O-SIL® fumed silica of approximately 5:1 and de-lumped through a No. 20 mesh screen. The other half portion of the spray dried particles was introduced into a secondary Ekato vertical dryer VPT-400. The spray dried particle/CAB-O-SIL° fumed silica blend was then added to the VPT-400 dryer and processed at 40° C., agitator rate @10 rpm, vessel pressure @ 30 mbar, nitrogen sweep rate @ 26 slpm, drying time 6 hours.

330.7 grams of the resulting dried material (94%) (referred to in the table below as “Preparation 1”) was discharged without manual intervention.

As a comparison to the above procedure and discharge result, Example 1 was repeated with the exception that CAB-O-SIL® fumed silica M5P colloidal silicon dioxide was not added during the drying step. In this comparison, out of a total of 346.8 grams of material that was subjected to secondary drying, only 30.59 grams (8.8%)(referred to in the table below as “Preparation 2—(control)”) of the resulting dried material was discharged without manual intervention.

| | | | Bulk | Tapped | | |
|---|---|---|---|---|---|---|
| | Bulk | Tapped | Specific | Specific | Carr | |
| | Density | Density | Volume | Volume | Index | Hausner |
| | (g/mL) | (g/mL) | (mL/g) | (mL/g) | (%) | Ratio |
| Preparation 1 | 0.17 | 0.25 | 5.8 | 4.0 | 31 | 1.45 |
| Preparation 2 | 0.07 | 0.19 | 14.1 | 5.3 | 62 | 2.65 |
| (control) | | | | | | |

The result of this was a yield increase from the Ekato drying tank discharge and the ability to reduce the further use silicon dioxide as an intra- or extra-granular excipient in the formulation. Furthermore, the necessary secondary drying time is reduced with silicon dioxide addition with little impact on the stability of the solid dispersion intermediate. Finally, the timing and method of silicon dioxide addition to the solid dispersion intermediate, in accordance with the described invention, improves the particle properties of the solid dispersion intermediate. The solid dispersion intermediate surface is essentially coated with very small silicon dioxide particles when added during the drying process. In contrast, when added only after the secondary drying process, the surface is less functionalized, and the silicon dioxide tends to exist as larger agglomerates next to the solid dispersion intermediate particles.

Carr's Index Testing Method:

Flowability was also evaluated using the Carr's Index calculated using bulk and tap density. Carr's Index is determined by the following equation:

C=100×(1−ρ_{B}/ρ_{T})

where ρ_{B} is equal to the freely settled bulk density and ρ_{T}is equal to the tapped density. In general, the lower the Carr's Index, the better the flowability of the substance. A powder having a lower Carr's Index can also be easier to compress into a tablet.
Bulk Density (freely settled) Method:
1. Obtain the tare weight of graduated cylinder and record.
2. Record total volume of cylinder (i.e., 10 cc or 100 cc).
3. Carefully add granulation or intermediate to a graduated cylinder being careful to handle as little as possible.
4. Obtain the weight of the cylinder with the sample and record.
5. Obtain the volume that the sample takes up in the cylinder.
6. To calculate the Bulk density take the weight of the sample and divide that by the volume recorded.

Bulk Density=Weight of Sample (g)/Volume of Sample (cc)

### Tapped Density Method:

1. Take the above Bulk Density sample in the cylinder and place in the Vankel Tap density.
instrument.
2. Tap for 2000 cycles.
3. Obtain the volume that the sample takes up in the cylinder and record.

Tapped Density=Weight of Sample (g)/Volume of Sample after 2000 Tap Cycles (cc)

### Hausner Ratio:

The Hausner Ratio is a number that is correlated to the flowability of a powder or granular material. It is calculated by the formula

H=ρ_{B}/ρ_{T}

where ρ_{B} is the freely settled bulk density of the powder, and ρ_{T} is the tapped bulk density of the powder. The Hausner Ratio is used in a wide variety of industries as an indication of the flowability of a powder. A Hausner Ratio greater than 1.25 is considered to be an indication of poor flowability. The Hausner ratio (H) is related to the Carr Index (C), another indication of flowability, by the formula

H=100/(100−C).

### EXAMPLE 2

Spray dried dispersions of dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]}) dicarbamate (2-1)

Elbasvir (dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate), which is shown below as Compound 2-1:

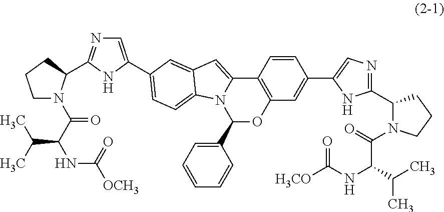 [SMILES: COC(=O)N[C@H](C(=O)N1CCC[C@H]1c1ncc(-c2ccc3c(c2)O[C@@H](c2ccccc2)n2c-3cc3cc(-c4cnc([C@@H]5CCCN5C(=O)[C@@H](NC(=O)OC)C(C)C)[nH]4)ccc32)[nH]1)C(C)C]

is described in U.S. Pat. No. 8,871,759. Details for preparing the compound are described in Example 223. Acetone, purified water, Vitamin E PGS (d-alpha tocopheryl polyethylene glycol 1000 succinate), Compound (2-1) and Hypromellose 2910 polymer were sequentially added and dissolved in a stainless steel tank equipped with agitation equipment at a temperature between 15 and 27° C. The weight proportion of the Compound (2-1)/Hypromellose 2910 polymer/Vitamin E PGS components in the blend was 20/70/10.

### Spray Drying

Using a Niro PSD-2 spray drying system with DPH Gas Disperser Type and Nozzle-Centering Device and Spraying Systems SK 76-17 (60 degree cone angle) pressure nozzle, and 12-inch outer diameter (O.D.) cyclone Product Collection, gas flow rate@ 450 kg/hr; Tᵢₙ@115° C.; Tₒᵤₜ@57° C.; Cond@−10° C.; feed pressure@485 PSI; feed rate@ 25 kg/hr, a solution feed filter of ≤250 μm, and a 75/25 (w/w) acetone/water for system warm-up and shutdown, the solvent blend described above containing Compound (2-1) was agitated for at least 30 minutes, pumped to the nozzle and spray-dried at standard operating conditions to create spray dried particles of the blend.

### Secondary Drying

The mixture of spray dried particles and CAB-O-SIL® fumed silica M5P colloidal silicon dioxide was subjected to secondary drying. A one half portion of the spray dried particles were blended for one minute with 1.5 wt % CAB-O-SIL® fumed silica M5P colloidal silicon dioxide in a volume ratio of particles to CAB-O-SIL® fumed silica of approximately 5:1 and de-lumped through a No. 20 mesh screen. The other half portion of the spray dried particles was introduced into a secondary Ekato vertical dryer VPT-400. The spray dried particle/CAB-O-SIL® fumed silica blend was then added to the VPT-400 dryer and processed at 40° C., agitator rate @10 rpm, vessel pressure @ 30 mbar, nitrogen sweep rate @ 26 slpm, drying time 6 hours.

The result of this was a yield increase from the Ekato drying tank discharge and the ability to reduce the further use silicon dioxide as an intra- or extra-granular excipient in the formulation. Furthermore, the necessary secondary drying time is reduced with silicon dioxide addition with little impact on the stability of the solid dispersion intermediate. Finally, the timing and method of silicon dioxide addition to the solid dispersion intermediate, in accordance with the described invention, improves the particle properties of the solid dispersion intermediate. The solid dispersion intermediate surface is essentially coated with very small silicon dioxide particles when added during the drying process. In contrast, when added only after the secondary drying process, the surface is less functionalized, and the silicon dioxide tends to exist as larger agglomerates next to the solid dispersion intermediate particles.

### EXAMPLE 3

### Tablet Composition

Following typical procedures for prepare a tablet dosage composition, and tablet having the following composition is formed:

| Component | Amount (mg/tablet) |
|---|---|
| Preparation 1 from | 150 |
| Example 1 | |
| Microcrystalline cellulose | 150 |
| Mannitol | 270 |
| Crospovidone | 24 |
| Magnesium stearate | 6 |
| Total | 600 |

What is claimed is:

1. A process for drying a spray dried dispersion, comprising:
a) providing a spray-dried dispersion comprising particles wherein the particles comprise an active agent and a polymer, the dispersion having an average particle diameter of less than about 100 μm;
b) blending an amount of silicon dioxide with the dispersion to form a dispersion-silicon dioxide blend, wherein the amount of silicon dioxide relative to the amount of dispersion is between about 0.5 and 2.0% by weight;
c) drying the dispersion-silicon dioxide blend with a secondary dryer, wherein the active agent is dimethyl ((2S,2′S)-((2S,2′S)-((6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-1,2-diyl))dicarbamate or dimethyl N,N′-([(6S)-6-phenylindolo[1,2-c][1,3]benzoxazine-3,10-diyl]bis{1H-imidazole-5,2-diyl-(2S)-pyrrolidine-2,1-diyl[(2S)-3-methyl-1-oxobutane-1,2-diyl]})dicarbamate.

2. A process of claim 1, wherein a second amount of silicon dioxide is added to the dispersion- silicon dioxide blend during step c), wherein the second amount of silicon dioxide relative to dispersion is between about 0.5 and 1.5% by weight

3. A process of claim 1 wherein the secondary dryer is an agitation dryer or vertical dryer.

4. A process of claim 3 wherein the vertical dryer is a vacuum contact dryer.

5. A process of claim 1, wherein the particles comprise an active agent, a polymer, and one or more surfactants.

6. A process for forming a pharmaceutical tablet, comprising:
a) providing a spray-dried dispersion comprising particles wherein the particles comprise an active agent and a polymer, the dispersion having an average particle diameter of less than about 100 μm;
b) blending an amount of silicon dioxide with the dispersion to form a dispersion-silicon dioxide blend, wherein the amount of silicon dioxide relative to the amount of dispersion is between about 0.5 and 2.0% by weight;
c) drying the dispersion- silicon dioxide blend with a secondary dryer;
d) adding pharmaceutically acceptable excipients to the dispersion- silicon dioxide blend; and
e) forming a tablet,
wherein the active agent is dimethyl ((2S,2′S)-((2S,2′S)-((6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-1,2-diyl))dicarbamate or dimethyl N,N′-([(6S)-6-phenylindolo[1,2-c][1,3 ]benzoxazine-3,10-diyl]bis{1H-imidazole-5,2-diyl-(2S)-pyrrolidine-2,1-diyl[(2S)-3-methyl-l-oxobutane-1,2-diyl]})dicarbamate.

7. A process of claim 6, wherein the active agent in step a) is a first active agent, and wherein a second active agent is added to the blend in step d).

8. A process of claim 7, wherein a third active agent is added is added to the blend in step d).

9. A process of claim 6, wherein the particles comprise an active agent, a polymer, and one or more surfactants.

10. A process of claim 6, which additionally includes granulating together the pharmaceutically acceptable excipients and the dispersion- silicon dioxide blend.
